SEQUENCE LISTING (62 sequences)

SEQ ID NO: 1 - protein, 13 aa, Artificial Sequence
  Description of Artificial Sequence Synthetic peptide
LQTTDNLLPMSPE

SEQ ID NO: 2 - protein, 13 aa, Artificial Sequence
  Description of Artificial Sequence Synthetic peptide
LQTTDNLLPMSPE

SEQ ID NO: 3 - protein, 19 aa, Artificial Sequence
  Description of Artificial Sequence Synthetic peptide
RRKYLKHRLIVVSNRQVDE

SEQ ID NO: 4 - protein, 19 aa, Artificial Sequence
  Description of Artificial Sequence Synthetic peptide
RRKYLKHRLIVVSNRQVDE

SEQ ID NO: 5 - protein, 7 aa, Artificial Sequence
  Description of Artificial Sequence Synthetic peptide
AVDGYVK

SEQ ID NO: 6 - protein, 7 aa, Artificial Sequence
  Description of Artificial Sequence Synthetic peptide
AVDGYVK

SEQ ID NO: 7 - protein, 14 aa, Artificial Sequence
  Description of Artificial Sequence Synthetic peptide
GYVPSVFIPISTIR

SEQ ID NO: 8 - protein, 14 aa, Artificial Sequence
  Description of Artificial Sequence Synthetic peptide
GYVPSVFIPISTIR

SEQ ID NO: 9 - protein, 20 aa, Artificial Sequence
  Description of Artificial Sequence Synthetic peptide
PHSPSDLLPMSPSVYAVLRE

SEQ ID NO: 10 - protein, 20 aa, Artificial Sequence
  Description of Artificial Sequence Synthetic peptide
PHSPSDLLPMSPSVYAVLRE

SEQ ID NO: 11 - protein, 19 aa, Artificial Sequence
  Description of Artificial Sequence Synthetic peptide
PESQEHPEADPGSAAPYLK

SEQ ID NO: 12 - protein, 19 aa, Artificial Sequence
  Description of Artificial Sequence Synthetic peptide
PESQEHPEADPGSAAPYLK

SEQ ID NO: 13 - protein, 20 aa, Artificial Sequence
  Description of Artificial Sequence Synthetic peptide
FICVTPTTCSNTIDLPMSPR

SEQ ID NO: 14 - protein, 20 aa, Artificial Sequence
  Description of Artificial Sequence Synthetic peptide
FICVTPTTCSNTIDLPMSPR

SEQ ID NO: 15 - protein, 15 aa, Artificial Sequence
  Description of Artificial Sequence Synthetic peptide
TSSAVWNSPPLQGAR

SEQ ID NO: 16 - protein, 15 aa, Artificial Sequence
  Description of Artificial Sequence Synthetic peptide
TSSAVWNSPPLQGAR

SEQ ID NO: 17 - protein, 13 aa, Artificial Sequence
  Description of Artificial Sequence Synthetic peptide
HTDDEMTGYVATR

SEQ ID NO: 18 - protein, 13 aa, Artificial Sequence
  Description of Artificial Sequence Synthetic peptide
HTDDEMTGYVATR

SEQ ID NO: 19 - protein, 14 aa, Artificial Sequence
  Description of Artificial Sequence Synthetic peptide
HSSPHQSEDEEDPR

SEQ ID NO: 20 - protein, 14 aa, Artificial Sequence
  Description of Artificial Sequence Synthetic peptide
HSSPHQSEDEEDPR

SEQ ID NO: 21 - protein, 11 aa, Artificial Sequence
  Description of Artificial Sequence Synthetic peptide
GADVFLEALAR

SEQ ID NO: 22 - protein, 12 aa, Artificial Sequence
  Description of Artificial Sequence Synthetic peptide
HSSPHQSEDEEE

SEQ ID NO: 23 - protein, 12 aa, Artificial Sequence
  Description of Artificial Sequence Synthetic peptide
HSSPHQSEDEEE

SEQ ID NO: 24 - protein, 19 aa, Artificial Sequence
  Description of Artificial Sequence Synthetic peptide
IADPEHDHTGFLTEYVATR

SEQ ID NO: 25 - protein, 19 aa, Artificial Sequence
  Description of Artificial Sequence Synthetic peptide
IADPEHDHTGFLTEYVATR

SEQ ID NO: 26 - protein, 19 aa, Artificial Sequence
  Description of Artificial Sequence Synthetic peptide
VADPDHDHTGFLTEYVATR

SEQ ID NO: 27 - protein, 19 aa, Artificial Sequence
  Description of Artificial Sequence Synthetic peptide
VADPDHDHTGFLTEYVATR

SEQ ID NO: 28 - protein, 8 aa, Artificial Sequence
  Description of Artificial Sequence Synthetic peptide
EYYSVHNK

SEQ ID NO: 29 - protein, 8 aa, Artificial Sequence
  Description of Artificial Sequence Synthetic peptide
EYYSVHNK

SEQ ID NO: 30 - protein, 11 aa, Artificial Sequence
  Description of Artificial Sequence Synthetic peptide
DLIGFGLQVAK

SEQ ID NO: 31 - protein, 20 aa, Artificial Sequence
  Description of Artificial Sequence Synthetic peptide
HVSISYDIPPTPGNTYQIPR

SEQ ID NO: 32 - protein, 20 aa, Artificial Sequence
  Description of Artificial Sequence Synthetic peptide
HVSISYDIPPTPGNTYQIPR

SEQ ID NO: 33 - protein, 13 aa, Artificial Sequence
  Description of Artificial Sequence Synthetic peptide
QVEYLDLDLDSGK

SEQ ID NO: 34 - protein, 13 aa, Artificial Sequence
  Description of Artificial Sequence Synthetic peptide
QVEYLDLDLDSGK

SEQ ID NO: 35 - protein, 15 aa, Artificial Sequence
  Description of Artificial Sequence Synthetic peptide
GFSFVATGLMEDDGK

SEQ ID NO: 36 - protein, 15 aa, Artificial Sequence
  Description of Artificial Sequence Synthetic peptide
GFSFVATGLMEDDGK

SEQ ID NO: 37 - protein, 17 aa, Artificial Sequence
  Description of Artificial Sequence Synthetic peptide
GFSFVATGLMEDDGKPR

SEQ ID NO: 38 - protein, 17 aa, Artificial Sequence
  Description of Artificial Sequence Synthetic peptide
GFSFVATGLMEDDGKPR

SEQ ID NO: 39 - protein, 22 aa, Artificial Sequence
  Description of Artificial Sequence Synthetic peptide
AENGLLMTPCYTANFVAPEVLK

SEQ ID NO: 40 - protein, 22 aa, Artificial Sequence
  Description of Artificial Sequence Synthetic peptide
AENGLLMTPCYTANFVAPEVLK

SEQ ID NO: 41 - protein, 18 aa, Artificial Sequence
  Description of Artificial Sequence Synthetic peptide
AYSFCGTVEYMAPEVVNR

SEQ ID NO: 42 - protein, 18 aa, Artificial Sequence
  Description of Artificial Sequence Synthetic peptide
AYSFCGTVEYMAPEVVNR

SEQ ID NO: 43 - protein, 16 aa, Artificial Sequence
  Description of Artificial Sequence Synthetic peptide
DSPGIPPSAGAHQLFR

SEQ ID NO: 44 - protein, 16 aa, Artificial Sequence
  Description of Artificial Sequence Synthetic peptide
DSPGIPPSAGAHQLFR

SEQ ID NO: 45 - protein, 19 aa, Artificial Sequence
  Description of Artificial Sequence Synthetic peptide
TPKDSPGIPPSAGAHQLFR

SEQ ID NO: 46 - protein, 19 aa, Artificial Sequence
  Description of Artificial Sequence Synthetic peptide
TPKDSPGIPPSAGAHQLFR

SEQ ID NO: 47 - protein, 11 aa, Artificial Sequence
  Description of Artificial Sequence Synthetic peptide
GSTAENAEYLR

SEQ ID NO: 48 - protein, 11 aa, Artificial Sequence
  Description of Artificial Sequence Synthetic peptide
GSTAENAEYLR

SEQ ID NO: 49 - protein, 19 aa, Artificial Sequence
  Description of Artificial Sequence Synthetic peptide
GSHQISLDNPDYQQDFFPK

SEQ ID NO: 50 - protein, 19 aa, Artificial Sequence
  Description of Artificial Sequence Synthetic peptide
GSHQISLDNPDYQQDFFPK

SEQ ID NO: 51 - protein, 22 aa, Artificial Sequence
  Description of Artificial Sequence Synthetic peptide
RPAGSVQNPVYHNQPLNPAPSR

SEQ ID NO: 52 - protein, 22 aa, Artificial Sequence
  Description of Artificial Sequence Synthetic peptide
RPAGSVQNPVYHNQPLNPAPSR

SEQ ID NO: 53 - protein, 31 aa, Artificial Sequence
  Description of Artificial Sequence Synthetic peptide
YSSDPTGALTEDSIDDTFLPVPEYINQSVPK

SEQ ID NO: 54 - protein, 31 aa, Artificial Sequence; identical to SEQ ID NO: 53
  Description of Artificial Sequence Synthetic peptide

SEQ ID NO: 55 - protein, 18 aa, Artificial Sequence
  Description of Artificial Sequence Synthetic peptide
AYSFCGTVEYMAPEVVNR

SEQ ID NO: 56 - protein, 18 aa, Artificial Sequence
  Description of Artificial Sequence Synthetic peptide
AYSFCGTVEYMAPEVVNR

SEQ ID NO: 57 - protein, 16 aa, Artificial Sequence
  Description of Artificial Sequence Synthetic peptide
DSPGIPPSAGAHQLFR

SEQ ID NO: 58 - protein, 16 aa, Artificial Sequence
  Description of Artificial Sequence Synthetic peptide
DSPGIPPSAGAHQLFR

SEQ ID NO: 59 - protein, 17 aa, Artificial Sequence
  Description of Artificial Sequence Synthetic peptide
GFSFVATGLMEDDGKPR

SEQ ID NO: 60 - protein, 17 aa, Artificial Sequence
  Description of Artificial Sequence Synthetic peptide
GFSFVATGLMEDDGKPR

SEQ ID NO: 61 - protein, 22 aa, Artificial Sequence
  Description of Artificial Sequence Synthetic peptide
AENGLLMTPCYTANFVAPEVLK

SEQ ID NO: 62 - protein, 22 aa, Artificial Sequence
  Description of Artificial Sequence Synthetic peptide
AENGLLMTPCYTANFVAPEVLK

(19) United States
(12) Patent Application Publication
(10) Pub. No.: US 2005/0074824 A1
(43) Pub. Date: Apr. 7, 2005

(54) Quantification and site-specific profiling of protein phosphorylation

(76) Inventors: Martyn Botfield, Boston, MA (US); David Friedman, Madison, CT (US)

Correspondence Address: FISH & NEAVE IP GROUP;ROPES & GRAY LLP, 1251 AVENUE OF THE AMERICAS FL C3, NEW YORK, NY, 10020-1105, US

(21) Appl. No.: 10/925,556

(22) Filed: Aug. 23, 2004

Related U.S. Application Data
(60) Provisional application No. 60/497,197, filed on Aug. 22, 2003.

(51) Int. Cl. G01N 33/53; G01N 33/567; C12Q 1/48

(52) U.S. Cl. 435/7.2; 435/15

(57) ABSTRACT

The invention provides methods and compositions for analyzing modification-mediated signaling pathways.

### CROSS-REFERENCE TO RELATED APPLICATIONS

This application claims the benefit of U.S. Provisional Application No. 60/497,197, filed Aug. 22, 2003, which is hereby incorporated by reference in its entirety for all purposes.

### FIELD OF THE INVENTION

This invention relates generally to the fields of biochemistry and molecular biology. More particularly, this invention relates to methods and compositions for analyzing modification-mediated signaling pathways.

### BACKGROUND OF THE INVENTION

Modification-mediated signaling pathways, such as kinase pathways, are common signaling pathways involved in cellular processes including, e.g., intracellular signaling, metabolism, cell division, cell growth, and cell differentiation. In addition, certain diseases, e.g. cancer, are associated with aberrations in specific signaling pathways. See, e.g., Ballif et al., “Molecular Mechanisms Mediating Mammalian Mitogen-activate Protein Kinases (MAPK) Kinase (MEK)-MAPK Cell Survival Signals,” Cell Growth & Differentiation 12: 397-408 (2001); Roux et al., “ERK and p38 MAPK-Activated Protein Kinases: a Family of Protein Kinases with Diverse Biological Functions,” Microbio. Molec. Biol. Rev. 68: 320-44 (2004). Accordingly, they are the subject of intense scientific study to understand their properties and to interfere with them pharmacologically.

In order to better understand signaling through modification-mediated signaling pathways, several methods have been developed to determine the modification state of a modifiable residue on their constituent proteins. These methods include, e.g., labeling with ³²P or anti-phosphotyrosine antibodies. These earlier approaches have significant disadvantages and mass spectrometry (MS) is increasingly becoming the method of choice for localization. For mass spectrometry, antibody pre-concentration of proteins from cellular lysates combined with high-resolution nano-scale chromatographic methods has greatly enhanced the selectivity and signal-to-noise achievable in automated stable-isotope dilution/tandem MS assays. These assays have the added advantage that it is possible to quantify the amount of total protein as well as the amount of both the modified and non-modified forms of the protein. For a description of such approaches, see, e.g., United States Patent Publication No. US2002/0192708; Tao et al., “Advances in quantitative proteomics via stable isotope tagging and mass spectrometry,” Curr. Opin. Biotech. 14: 110-18 (2003); Zhou et al., “Quantitative proteome analysis by solid-phase isotope tagging and mass spectrometry,” Nature Biotech. 19: 512-15 (2002); and Havlis et al., “Absolute quantification of proteins in solutions and in polyacrylamide gels by mass spectrometry,” Anal. Chem. 76: 3029-36 (2004), each of which is hereby incorporated by reference in its entirety for all purposes.

These methods provide information only about the modification states of particular proteins, but they do not provide information about the complex interactions between different modification-mediated signaling pathways in a cell and/or between signaling pathways and compounds that modulate their activity. Accordingly, there remains a need for methods and compositions to understand these aspects of modification-mediated signaling pathways.

### SUMMARY OF THE INVENTION

The present invention is based, at least in part, on the discovery that MS-based methods for monitoring modification states on proteins in modification-mediated signaling pathways can be used to simultaneously monitor multiple modification sites on one or more proteins. These methods also can be used to monitor different types of modification simultaneously and to calculate and compare the magnitude of drive through different signaling pathways.

In some embodiments, the invention provides a method for identifying a target protein of an inhibitor of a modification-mediated signaling pathway comprising the steps of: (a) contacting said inhibitor with a biological sample expressing at least two proteins in said signaling pathway; (b) determining the fractional occupancy of modification of at least one modifiable residue in each of said at least two proteins; (c) comparing the fractional occupancy of modification in (b) to the fractional occupancy of modification of said at least one modifiable residue in each of said at least two proteins in the absence of said inhibitor; and (d) identifying an upstream protein and its immediate downstream protein in said at least two proteins in said signaling pathway, wherein the fractional occupancy of said upstream protein evidences modification by said signaling pathway in the presence of said inhibitor and wherein the fractional occupancy of its immediate downstream protein evidences reduced modification by said signaling pathway in the presence of said inhibitor; wherein said upstream protein is a target protein of said inhibitor. In some embodiments, the modification-mediated signaling pathway is a kinase pathway. In some embodiments, the kinase pathways comprises a protein selected from the group consisting of: RSK, ERK, cMet, Glycogen Synthase, STAT1-5, Histone H3, INCEP, and EGFR. In some embodiments, the biological sample is selected from the group consisting of: cultured cells, harvested tissues, and clinical samples.

In some embodiments, the invention provides a method for identifying an optimal protein target for modulation in a modification-mediated signaling pathway comprising at least two proteins, the method comprising the steps of: (a) determining the fractional occupancy of modification of each of said at least two proteins in a biological sample; (b) quantifying the amount of each of said two proteins; and (c) calculating the drive product and the drive ratio for each of said two proteins; and (d) wherein the protein with the highest drive product and the lowest drive ratio is the optimal protein target for modulation; or if two proteins have similar drive products, then the protein with the lower drive ratio therebetween is the optimal protein target. In some embodiments, the modification-mediated signaling pathway is a kinase pathway. In some embodiments, the kinase pathway comprises a protein selected from the group consisting of: RSK, ERK, cMet, Glycogen Synthase, STAT1-5, Histone H3, INCEP, and EGFR. In some embodiments, the modulation is inhibition. In some embodiments, the biological sample is selected from the group consisting of: cultured cells, harvested tissues, and clinical samples.

In some embodiments, the invention provides a method of determining which of at least two modification-mediated signaling pathways is the target of a modulator comprising the steps of: (a contacting said modulator with a biological sample having said at least two pathways, wherein each of said pathways causes at least one distinguishable modification on a protein; (b) determining the fractional occupancy of said at least one distinguishable modification caused by each pathway; and (c) comparing the fractional occupancy of the distinguishable modifications in (b) to the fractional occupancy of the distinguishable modifications in the absence of said modulator; wherein alteration of the amount of a distinguishable modification in the presence of said modulator as compared to in the absence of said modulator indicates that said modulator acts on the signaling pathway that causes said distinguishable modification. In some embodiments, the modification-mediated signaling pathways are kinase pathways. In some embodiments, the kinase pathways comprise proteins selected from the group consisting of: RSK, ERK, cMet, Glycogen Synthase, STAT1-5, Histone H3, INCEP, and EGFR. In some embodiments, the modulator is an inhibitor. In some embodiments, the biological sample is selected from the group consisting of: cultured cells, harvested tissues, and clinical samples.

In some embodiments, the invention provides a method of identifying the dominant driver between at least two modification-mediated signaling pathways, wherein each of said signaling pathways causes at least one distinguishable modification on a protein, said method comprising the steps of: (a) determining the fractional occupancy of said at least one distinguishable modification caused by each signaling pathway in a biological sample in which said signaling pathways are operative; (b) quantifying the amount of said protein; (c) calculating the drive product and the drive ratio for said protein for said at least one distinguishable modification caused by each signaling pathway; and (d) calculating the difference between the extent of said distinguishable modifications of (c) and the extent of each of said distinguishable modifications in the absence of said treatment; wherein the signaling pathway which results in a modification with the highest drive product and the lowest drive ratio is the dominant driver; or if two signaling pathways have similar drive products, then the signaling pathway with the lower drive ratio therebetween is the dominant driver. In some embodiments, the distinguishable modifications occur at two separately modifiable residues of said protein. In some embodiments, the modification-mediated signaling pathway is a kinase pathway. In some embodiments, then distinguishable modifications are both phosphorylation. In some embodiments, then biological sample is selected from the group consisting of: cultured cells, harvested tissues, and clinical samples.

In some embodiments, the invention provides a method of determining which of at least two modification-mediated signaling pathways is more sensitive to a modulator comprising the steps of: (a) determining the fractional occupancy of modification of said at least one protein in each of said at least two modification-mediated signaling pathways in a biological sample; (b) quantifying the amount of said at least one protein per cell; (c) calculating the drive product and drive ratio for said at least one protein; and (d) calculating the difference between the drive product and drive ratio of (c) and the drive product and drive ratio of said protein in the absence of said modulator; wherein the protein with the highest difference in the drive product and the lowest difference in the drive ratio is more sensitive therebetween to said modulator; or if two signaling pathways have similar differences in the drive products, then the signaling pathway with the lowest difference in the drive ratio is the more sensitive therebetween to said modulator. In some embodiments, then modification-mediated signaling pathway is a kinase pathway. In some embodiments, then distinguishable modifications are both phosphorylation. In some embodiments, the biological sample is selected from the group consisting of: cultured cells, harvested tissues, and clinical samples.

In some embodiments, the invention provides a method of monitoring the progress in a patient of therapy using a modulator of a protein comprising at least one modifiable residue, said method comprising the steps of: (a) exposing said patient to said compound; (b) obtaining a biological sample comprising said protein from said patient; (c) determining the extent of modification of said at least one modifiable residue is said cells; and (d) comparing the extent of modification of said at least one modifiable residue to the extent of modification of said at least one modifiable residue in the absence of said compound. In some embodiments, the modification-mediated signaling pathway is a kinase pathway. In some embodiments, the kinase pathway comprises a protein selected from the group consisting of: RSK, ERK, cMet, Glycogen Synthase, STAT1-5, Histone H3, INCEP, and EGFR. In some embodiments, then modulator is an inhibitor. In some embodiments, the biological sample is selected from the group consisting of: a blood sample or a tumor biopsy.

In some embodiments, the invention provides a method of determining which of at least two modulators of a modification-mediation signaling pathway is more useful as a treatment for a condition characterized by aberrant signaling through said pathway comprising: (a) separately exposing at least two biological samples characterized by aberrant signaling through said pathway to each of said at least two modulators, wherein said biological samples comprise a protein that is modified by said modification-mediated signaling pathway; (b) separately determining the extent of modification of said protein; (c) separately quantifying the amount of said protein per cell; (d) calculating the drive products and drive ratios for said protein in the presence of each of said at least two modulators; and (e) calculating the differences between the drive products and drive ratios of (d) to the drive product and drive ratio for said protein in said cell in the absence of said modulator; wherein the modulator that is associated with the highest difference in drive product and the lowest difference in drive ratio in (e) is more useful therebetween as a treatment for said condition. In some embodiments, the modification-mediated signaling pathway is a kinase pathway. In some embodiments, then kinase pathway comprises a protein selected from the group consisting of: RSK, ERK, cMet, Glycogen Synthase, STAT1-5, Histone H3, INCEP, and EGFR. In some embodiments, the modulators are inhibitors. In som embodiments, the biological sample is selected from the group consisting of: cultured cells, harvested tissues, and clinical samples.

In some embodiments, the invention provides a composition comprising at least one oligopeptide selected from SEQ ID NO: 1-54, wherein each of said polypeptides comprises at least one modification that renders it distinguishable by MS from the corresponding non-modified oligopeptide. In some embodiments, the modification is at least one amino-acid residue comprising ¹³C and/or ¹⁵N. In some embodiments, then modified amino-acid residues comprise an affinity tag.

### BRIEF DESCRIPTION OF THE DRAWINGS

FIG. 1 shows the fractional occupancy of phosphorylation of the S360 residue in control mixtures of recombinant p90 Rsk.

FIG. 2. shows the phosphorylation-site-specific effect of a kinase inhibitor on p90 Rsk in HT-29 cells.

FIG. 3 shows fractional occupancy of phosphorylation of S360, S380, S221, and T573 of p90 Rsk in an SK-Mel-28 tumor over time.

### DETAILED DESCRIPTION OF THE INVENTION

The present invention provides methods of identifying and analyzing modification-mediated signaling pathways. The invention relates to the application of an assay that we designate ITIS (Isotope-tagged internal standard), to interrogate and quantify the site-specific modification of target proteins using LC/MS/MS. This approach is based on using tryptic digests of the protein combined with the additions of known quantities of mass-tagged internal standards (e.g., ¹³C-, ¹⁵N-, or affinity-tagged synthetic peptides corresponding to the tryptic fragments containing the modifiable sites of interest) followed by LC/MS/MS analysis. These “heavy” mass-tagged standards co-purify through the chromatographic steps and permit quantification of corresponding “light” peptides by MS/MS. Thus, this method supports simultaneous quantification of multiple modifiable residues in a target protein. ITIS is compatible with cell, tissue, and clinical isolates. In addition, it provides both absolute percent modification as well as absolute quantity of the modified protein.

In some embodiments, the invention provides methods for identifying which protein in a modification-mediated signaling pathway is a target of an inhibitor. Generally, the inhibitor is known to be an inhibitor that acts on the particular modification-mediated signaling pathway assayed. However, one of skill in the art will recognize that this method could also be used as to screen multiple compounds for activity against a particular pathway, e.g., to identify a set of compounds that inhibit a particular step or individual compounds that inhibit each step in a particular pathway.

As used herein, a “modification-mediated signaling pathway” is a signaling pathway in which signaling is mediated by sequential chemical modification of one or more proteins in the signaling pathway. Such chemical modifications include, e.g., phosphorylation on amino-acid residues (e.g., serine, threonine, or tyrosine), ubiquitination, and famesylation. Such signaling pathways are involved in a wide variety of phenotypes.

As used herein, an “upstream protein” and a “downstream protein” in a modification-mediated signaling pathway are adjacent proteins in the pathway such that the upstream protein signals through the downstream protein. A given signaling pathway is composed of multiple, overlapping upstream and downstream protein pairs. One of skill in the art will recognize that a given protein can be an upstream or downstream protein with respect to more than one other protein. For example, a protein that integrates the signal from two separate modification-mediated signaling pathways is a downstream protein with respect to each of two upstream proteins in those two pathways.

The methods of the invention involve analysis of proteins comprising “modifiable residues.” As used herein, a modifiable residue is an amino acid that is capable of being modified by the action of a modification-mediated signaling pathway. One of skill in the art will recognize that a modifiable residue may be modified or non-modified depending, e.g., on whether the corresponding modificaition-mediated signaling pathway is active. Examples of modifiable residues include, but are not limited to, serine, threonine, and tyrosine.

In some embodiments, the invention provides a method for identifying an optimal protein target for modulation in a modification-mediated signaling pathway. Such an “optimal protein target for modulation” is, e.g., a protein in the signaling pathway that has properties that make it a particularly appropriate for pharmacological intervention.

In some embodiments, the invention provides a method of determining which of at least two modification-mediated signaling pathways is the target of a particular modulator.

As used herein, a “modulator” of a modification-mediated signaling pathway includes compounds that impact the pathway in any way, including, e.g., increasing, decreasing, and blocking signaling. A modulator can act at any step in the modification-mediated signaling pathway. Examples of such modulators include, but are not limited to, kinase inhibitors and kinase activators.

In some embodiments, the invention provides a method of identifying the “dominant driver” through a protein between at least two modification-mediated signaling pathways. A dominant driver is the signaling pathway that has the greater (or greatest where a protein integrates the signals of at least three modification-mediated signaling pathways) impact on the modification state of the protein. One of skill in the art will recognize that the dominant driver will have greater impact on, e.g., the signal that is processed through a protein that integrates the signals from two or more competing modification-mediated signaling pathways.

As used herein, “fractional occupancy” of a modifiable residue on a protein is a measure of the amount of the protein wherein that modifiable residue is modified as compared to the amount of the protein wherein the same modifiable residue is not modified. Accordingly, fractional occupancy can be measured or expressed in a various ways known to one of skill in the art. For example, fractional occupancy can be expressed as the amount of a protein in which a modifiable residue is modified over the total amount of protein or as the amount of a protein in which a modifiable residue is modified over the amount of the protein in which a modifiable residue is not modified.

As used herein, “drive” is a quantitative measure of the flux through a particular protein component of a modification-mediated signaling pathway. Drive has two components—“drive product” and “drive ratio.” The drive product is the arithmetic product of fractional occupancy and the copy number of the corresponding protein. The drive ratio is the arithmetic ratio of the copy number of the protein divided by the fractional occupancy. The amount of protein is determined by MS in the same experiment in which fractional occupancy is determined and this number is normalized to the amount of biological material used in the assay to obtain the copy number of the protein. One of skill in the art will recognize that copy number of the protein can be normalized to a number of variables depending on the biological sample used, including, e.g., mass, volume, and cell number.

In some embodiments, the methods of the invention involve comparisons between two values, e.g., between two drive products or drive ratios. As used herein, two such values are “similar” when it is not possible to distinguish therebetween within the limitations of a given means. For example, two values will be similar if they are not statistically distinguishable from each other.

In some embodiments, the invention provides a method of determining which of at least two modification-mediated signaling pathways is more sensitive to a modulator known to act on the pathways. For example, this method could be used to identify a modulator that could be advantageously used at a lower concentration. Such a modulator could be used in treatment and might have reduced toxic effects.

In some embodiments, the invention provides a method of determining which of at least two modulators of a modification-mediation signaling pathway is more useful as a treatment for a condition characterized by aberrant signaling through said signaling pathway. One of skill in the art can identify signaling pathways that exhibit aberrant signaling. For example, certain diseases are associated with excessive signaling through the RSK and MSK signaling pathways.

In some embodiments, the invention provides compositions of oligopeptides useful for analyzing the modification mediated signaling through particular proteins. These include, e.g., RSK, ERK, cMet, Glycogen Synthase, STAT1-5, Histone H3, INCEP, and EGFR. One of skill in the art will recognize that these oligopeptides are modified such that they can be distinguished in LC/MS/MS from the corresponding tryptic peptides obtained from a biological sample. For example, the oligopeptides in the compositions may comprise one or more amino acids with heavy atoms (e.g., ¹³C or ¹⁵N) or an affinity tag. The compositions of the invention generally comprise pairs of oligopeptides that are specific for the modifed and non-modified forms the corresponding tryptic peptide of a protein to be analyzed (e.g., SEQ ID NOs: 1 and 2, which are the un-phosphorylated and phosphorylated oligopeptides, respectively). In some embodiments, the compositions comprise at least two, e.g., three, four, and five, of the pairs of oligonucleotides needed to analyze multiple modifiable residues in a given protein. Table 1 includes a listing of certain oligopeptides of the invention as well as the protein and its corresponding modifiable residue for which they can used to determine the fractional occupancy and drive. Except for SEQ ID NOs: 21 and 30, which are control oligopeptides used to ascertain changes in the entire protein without complications introduced via phosphopeptides, the oligopeptides in Table 1 are paired as non-phosphorylated and phosphorylated forms.

TABLE 1

Sequences of Oligopeptides in Compositions
of the Invention

| | | |
|---|---|---|
| Protein | | |
| Interro- | | SEQ ID |
| gated | Oligopeptide Amino-acid Sequence* | NO: |

| | | |
|---|---|---|
| Stat 1 | LQTTDNLLPMsPE | 1 |
| Stat 1 | LQTTDNLLPMs(PO₄)PE | 2 |
| Stat 2 | RRKyLKHRLIVVSNRQVDE | 3 |
| Stat 2 | RRKy(PO₄)LKHRLIVVSNIRQVDE | 4 |
| Stat 5B | AVDGyVK | 5 |
| Stat 5B | AVDGy(PO₄)VK | 6 |
| Stat 4 | GyVPSVFIPISTIR | 7 |
| Stat 4 | Gy(PO₄)VPSVFIPISTIR | 8 |
| Stat 4 | PHSPSDLLPMsPSVYAVLRE | 9 |
| Stat 4 | PHSPSDLLPMs(PO₄)PSVYAVLRE | 10 |
| Stat 3 | PESQEHPEADPGSAAPyLK | 11 |
| Stat 3 | PESQEHPEADPGSAAPy(PO₄)LK | 12 |
| Stat 3 | FICVTPTTCSNTIDLPMsPR | 13 |
| Stat 3 | FICVTPTTCSNTIDLPMs(PO₄)PR | 14 |
| TNCEP | TssAVWNSPPLQGAR | 15 |
| INCEP | Ts(PO₄)s(PO₄)AVWNSPPLQGAR | 16 |
| MK14 | HTDDEMtGyVATR | 17 |
| MK14 | HTDDEMt(PO₄)Gy(PO₄)VATR | 18 |
| Glycogen | HSsPHQsEDEEDPR | 19 |
| synthase | | |
| Glycogen | HSs(PO₄)PHQs(PO₄)EDEEDPR | 20 |
| synthase | | |
| Glycogen | GADVFLEALAR | 21 |
| synthase | | |
| Glycogen | HSsPHQsEDEEE | 22 |
| synthase | | |
| Glycogen | HSs(PO₄)PHQs(PO₄)EDEEE | 23 |
| synthase | | |
| ERK 1/2 | IADPEHDHTGFLtEyVATR | 24 |
| ERK 1/2 | IADPEHDHTGFLt(PO₄)EY(PO₄)VATR | 25 |
| ERK 1/2 | VADPDHDHTGFLtEyVATR | 26 |
| ERK 1/2 | VADPDHDHTGFLt(PO₄)Ey(PO₄)VATR | 27 |
| cMet | EyySVHNK | 28 |
| cMet | Ey(PO₄)y(PO₄)SVHNK | 29 |
| GAB1 | HVSISyDIPPTPGNTYQIPR | 31 |
| GAB1 | HVSISy(PO₄)DIPPTPGNTYQIPR | 32 |
| GAB1 | QVEyLDLDLDSGK | 33 |
| GAB1 | QVEy(PO₄)LDLDLDSGK | 34 |
| RSK | GFsFVATGLMEDDGK | 35 |
| RSK | GFs(PO₄)FVATGLMEDDGK | 36 |
| RSK | GFsFVATGLMEDDGKPR | 37 |
| RSK | GFs(PO₄)FVATGLMEDDGKLPR | 38 |
| RSK | AENGLLMtPCYTANFVAPEVLK | 39 |
| RSK | AENGLLMt(PO₄)PCYTANFVAPEVLK | 40 |
| RSK | AYsFCGTVEYMAPEVVNR | 41 |
| RSK | AYs(PO₄)FCGTVEYMAPEVVNR | 42 |
| RSK | DsPGIPPSAGAHQLFR | 43 |
| RSK | Ds(PO₄)PGIPPSAGAHQLFR | 44 |
| RSK | tPKDsPGIPPsAGAHQLFR | 45 |
| RSK | t(PO₄)PKDs(PO₄)PGIPPs(PO₄)AGAHQLFR | 46 |
| EGFR | GSTAENAEyLR | 47 |
| EGFR | GSTAENAEy(PO₄)LR | 48 |
| EGFR | GSHQISLDNPDyQQDFFPK | 49 |
| EGFR | GSHQISLDNPDy(PO₄)QQDFFPK | 50 |
| EGFR | RPAGSVQNPVyHNQPLNPAPSR | 51 |
| EGFR | RPAGSVQNPVy(PO₄)HNQPLNPAPSR | 52 |
| EGFR | YSSDPTGALTEDSIDDTFLPVPEyINQSVPK | 53 |
| EGFR | YSSDPTGALTEDSIDDTFLPVPEy(PO₄)INQSVPK | 54 |

*The modifiable residue is in lowercase. “PO₄” indicates a phorphoryl group on the immediately preceding modifiable residue in the oligopeptide.

The methods of the invention may be performed with any biological sample that comprises an operative modification-mediated signaling pathway. Typically, the methods are performed with one or more purified proteins, a cell culture, a tissue sample, or a clinical sample. The clinical sample can be, e.g., a biopsy from a mammal, e.g., a human. A biopsy can be, e.g., a blood sample or tumor tissue.

Although methods and materials similar or equivalent to those described herein can be used in the practice or testing of the invention, specific methods and materials that may be used in the invention are described below. While the materials, methods and examples exhibit some embodiments of the invention, they are illustrative only, and are not intended to limit the full scope of the invention. Other features and advantages of the invention will be apparent from the description and from the claims.

### EXAMPLES

### Example 1

### Application of ITIS to p90 Rsk

We adapted the ITIS system to quantify fractional phosphorylation of four phosphorylatable sites in p90 Rsk: S221, S360, S280, and T573. Two of these sites are putative Rsk-modification sites (S360 and T573), a third is an Erk-dependent, Rsk-autophorphorylation site (S380), and the fourth (S221) is a PDKI-modification site that is important for docking Rsk with other associated proteins.

Heavy Mass-tagged Standards for p90 Rsk. We first obtained synthetic oligopeptides corresponding to the Rsk tryptic peptides that contained one ¹³C- and ¹⁵N-labeled amino acid each. The sequences and compositions of these oligopeptides as well as the p90 Rsk phosphorylation site that they contain are shown in Table 2.

TABLE 2

Mass-tagged oligopeptides used for ITIS
analysis of p90 Rsk

| | | | | |
|---|---|---|---|---|
| Peptide | | SEQ | Mass | |
| Standard | | ID | Tag | Query |
| ID | Sequence¹ | NO: | (amu) | Site |

| | | | | |
|---|---|---|---|---|
| A | AYSFcGTVEYMAPEVV*NR | 55 | +6 | S221 |
| B | AYsFcGTVEYMAPEVV*NR | 56 | +6 | s221 |
| C | DSPGTPPSAGAHQL*FR | 57 | +7 | S360 |
| D | DsPGIIPPSAGAHQL*FR | 58 | +7 | s360 |
| E | GFSFVATGLMEDDGKP*R | 59 | +6 | S380 |
| F | GFsFVATGLMEDDGKP*R | 60 | +6 | s380 |
| G | AENGLLMTPcYTANFVAPEVL*K | 61 | +7 | T573 |
| H | AENGLLMtPcYTANFVAPEVL*K | 62 | +7 | t573 |

¹“S” = serine; “s” = phosphoserine; “T” = threonine; “t” = phosphothreonine;
*indicates the heavy amino acid that is the mass tag.

The Assay System. We performed the assay by isolating Rsk from cells or tissues by immunoprecipitation with a pan-Rsk antibody precoupled to agarose beads (SantaCruz; sc-231). We then washed the precipitates 6 times with PBS containing 1% NP-40 and then once with distilled water prior to elution of the beads with 0.1% SDS-PAGE buffer. The samples were then heated for 10 minutes at 70° C., spun to remove the beads, and loaded on mini-slab geles (InVitrogen). The p90 Rsk bands (estimated by MW and recominant standards) were excised and digested with trypsin using standard protocols. The tryptic peptides were then extracted and mixed with a known amount of the oligopeptides shown in Table 1. The samples were then analyzed by LC/MS/MS as described in Yates, “Mass Spectral Analysis In Proteomics,” Annu. Rev. Biophys. Bimolec. Structure 33: 297-316 (2004).

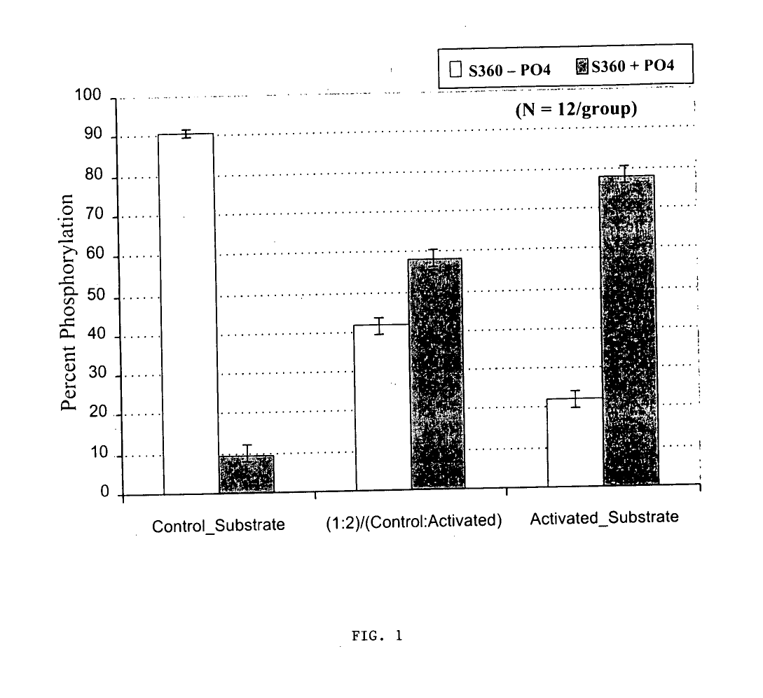

Accuracy of Measurement of Occupancy in p90 Rsk. To confirm that the assay could accurately detect changes in p90 Rsk phosphorylation, we mixed purified samples of non-activated (S360) and activated (pS360) p90 Rsk at defined ratios to create different percentages of phosphorylation. We purified recombinant p90 Rsk expressed in baculovirus and activated it in vitro using purified Erk kinase. We then mixed the activated p90 Rsk with non-activated p90 Rsk to produce three percentages of phosphorylation: 100%, 66%, and non-activated. We then evaluated these samples using the assay system described above. The results of this experiment confirmed that the phosphorylation occupancy at S360 was accurately determined (FIG. 1).

Simultaneous Measurement of Occupancy at S221, S360, S380, and T573. We next confirmed that the assay could be used to simultaneously evaluate the phosphorylation occupancy at all four p90 Rsk phosphorylation sites in a wide range of cell types, including MiPaCa tumors (tissue), MDAMB468 cells (2.5×10⁷ cells), PC3 (p13) cells (2.4×10⁶ cells), MiPaCa cells (4.4×10⁷ cells), and ZR-75 tumors (tissue). The results of this experiment confirmed that the phosphorylation occupancy at all four sites can be simultaneously determined (Table 3).

TABLE 3
Phosphorylation Occupancy at Each Modifiable Residue of p90 Rsk

| | S360 | S380 | S221 | T573 |
|---|---|---|---|---|
| MiPaCa tumors | 28% | 1.1% | 68% | 1.7% |
| MiPaCa cells | 25% | 0.8% | 85% | 0.9% |
| MDAMB468 cells | 35% | 6% | 84% | 1.1% |
| PC3 (p13) cells | 1.6% | 0.2% | 65% | 1.3% |
| ZR-75 tumors | 38% | 4% | 64% | 2.6% |

### Example 2

### Application of ITIS to Analysis of Inhibitors

We next tested whether the ITIS system could be used to screen for kinase modulators, including modulators that act at a specific modifiable residue in a protein.

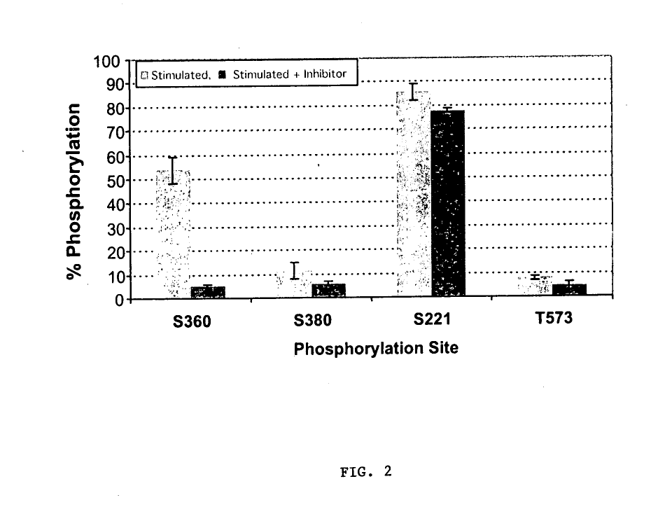

We grew serum-stimulated HT-29 cells in the presence and absence of 100 nM of a p90 Rsk inhibitor and performed the ITIS analysis on p90 Rsk from these cells as described in Example 1. We observed that the inhibitor had a general effect, reducing phosphorylation detectably at all four sites, but that phosphorylation of the S360-site was most dramatically reduced (FIG. 2). These results indicated that the ITIS assay may be used to screen for kinase modulators, including site-specific kinase modulators; to identify the site(s) of action of modulators; and/or to monitor the effect of treatment with modulators.

### Example 3

### Use of ITIS for Analysis of Tumor Progression

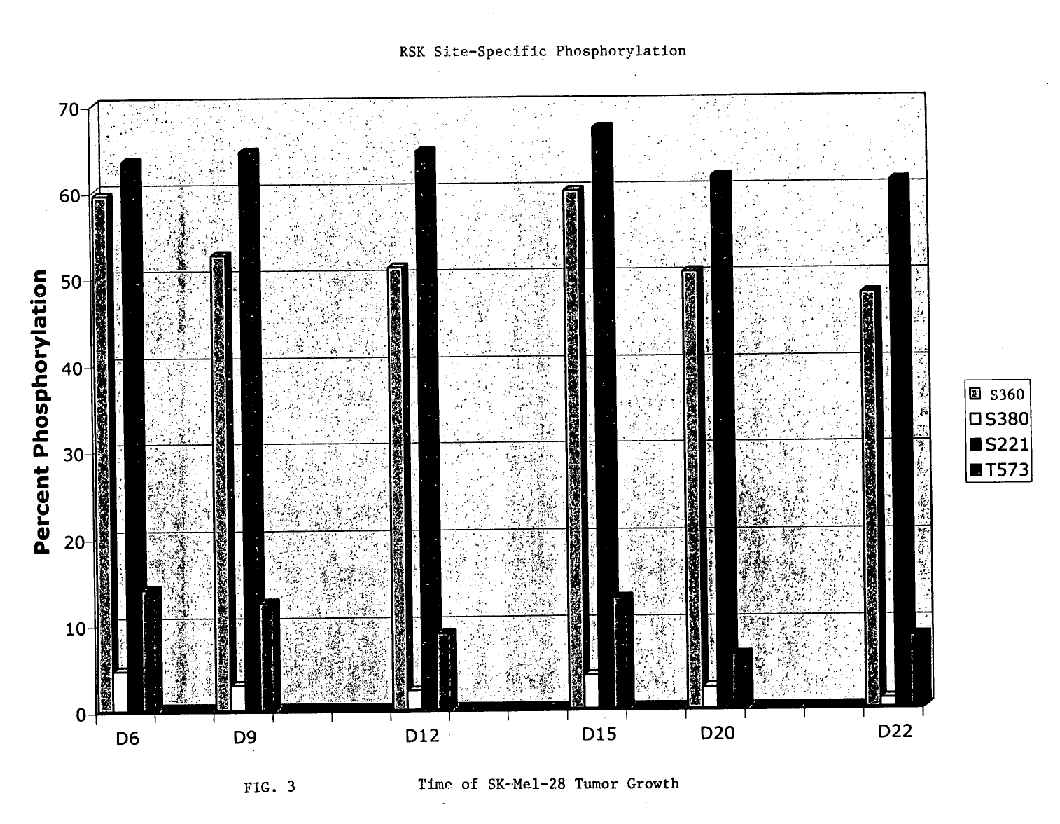

We used the ITIS assay system to monitor the phosphorylation occupancy at all four p90 Rsk sites in an SK-Mel-28 tumor over time. We performed the ITIS analysis as described in Example 1 on p90 Rsk from an SK-Mel-28 tumor at days 6, 9, 12, 15, 20 and 22 after induction. We observed that there is some fluctuation in the phosphorylation site occupancy at all four p90 Rsk sites over time (FIG. 3). These results indicate that the ITIS assay may be used to monitor modification site occupancy in vivo as an indicator of, e.g., tumor progression and/or effectiveness of treatment with a modulator.

### Example 4

### Analysis of Drive Through Erk in vitro and in vivo

We observed that ZR-75 breast cancer cells, which have increase drive through Erk, are resistant to an inhibitor when grown in vitro (IC50=2650 nM), but highly sensitive when grown as ectopic tumors in rats (dosage of 150 mg/kg). We used the ITIS assay system to compare the Erk drive in vitro and in vivo to determine whether this phenotype is different between the models and, accordingly, whether this may contribute to the difference in inhibitor resistance. As a control, we also tested MiPaCa cells in vitro and in vivo. The cells were harvested as cultured cells or as solid tumor tissue derived from a mouse xenograft model. We assayed the fractional occupancy of phosphorylation on p90 Rsk residues S221, S360, S280, and T573 as described in Example 1. Phosphorylation of the S360 residue is Erk-dependent. The results of this experiment are shown in Table 4.

TABLE 4
Percentage Phosphorylation at RSK Sites in vivo and in vitro

| | S360 | S380 | S221 | T573 | Replicates |
|---|---|---|---|---|---|
| ZR-75 Cells | 12.66 | 1.14 | 46.63 | 4.80 | 3 |
| ZR-75 Tumors | 47.10 | 4.90 | 73.00 | 2.80 | 43 |
| MiPaCa Cells | 25.0 | 0.84 | 85.5 | 0.94 | 3 |
| MiPaCa Tumors | 28.0 | 1.1 | 67.9 | 1.7 | 25 |

These data revealed that in one cell type, MiPaCa cells, there is no difference in the RSK phosphorylation fractional occupancy when cultured cells are compared to tumors. In contrast, the RSK phosphotype of ZR-75 cells in vitro was considerably different from the RSK phosphorylation fractional occupancy derived from xenograft tumors. This was especially noticeable at the S360 site where the occupancy in vitro is only 12.66%, as compared to in vivo where it is 47.10%. A similar difference was also seen at the S380 site where the occupancy in vivo is about four-fold higher (1.14 vs. 4.90). This difference indicated that there is a significantly higher ERK drive when the ZR-75 cells are implanted into a host animal and grow as a solid tumor compared to the ERK drive observed from cells cultured in vitro.

### Other Embodiments

Other embodiments are within the following claims.

1. A method for identifying a target protein of an inhibitor of a modification-mediated signaling pathway comprising the steps of:
a) contacting said inhibitor with a biological sample expressing at least two proteins in said signaling pathway;
b) determining the fractional occupancy of modification of at least one modifiable residue in each of said at least two proteins;
c) comparing the fractional occupancy of modification in (b) to the fractional occupancy of modification of said at least one modifiable residue in each of said at least two proteins in the absence of said inhibitor; and
d) identifying an upstream protein and its immediate downstream protein in said at least two proteins in said signaling pathway, wherein the fractional occupancy of said upstream protein evidences modification by said signaling pathway in the presence of said inhibitor and wherein the fractional occupancy of its immediate downstream protein evidences reduced modification by said signaling pathway in the presence of said inhibitor;
wherein said upstream protein is a target protein of said inhibitor.

2. The method of claim 1, wherein said modification-mediated signaling pathway is a kinase pathway.

3. The method of claim 2, wherein said kinase pathway comprises a protein selected from the group consisting of: RSK, ERK, cMet, Glycogen Synthase, STAT1-5, Histone H3, INCEP, and EGFR.

4. The method of claim 1, wherein said biological sample is selected from the group consisting of: cultured cells, harvested tissues, and clinical samples.

5. A method for identifying an optimal protein target for modulation in a modification-mediated signaling pathway comprising at least two proteins, the method comprising the steps of:
a) determining the fractional occupancy of modification of each of said at least two proteins in a biological sample;
b) quantifying the amount of each of said two proteins;
c) calculating the drive product and the drive ratio for each of said two proteins; and
d) wherein the protein with the highest drive product and the lowest drive ratio is the optimal protein target for modulation; or if two proteins have similar drive products, then the protein with the lower drive ratio therebetween is the optimal protein target.

6. The method of claim 5, wherein said modification-mediated signaling pathway is a kinase pathway.

7. The method of claim 6, wherein said kinase pathway comprises a protein selected from the group consisting of: RSK, ERK, cMet, Glycogen Synthase, STAT1-5, Histone H3, INCEP, and EGFR.

8. The method of claim 5, wherein said modulation is inhibition.

9. The method of claim 5, wherein said biological sample is selected from the group consisting of: cultured cells, harvested tissues, and clinical samples.

10. A method of determining which of at least two modification-mediated signaling pathways is the target of a modulator comprising the steps of:
a) contacting said modulator with a biological sample having said at least two pathways, wherein each of said pathways causes at least one distinguishable modification on a protein;
b) determining the fractional occupancy of said at least one distinguishable modification caused by each pathway; and
c) comparing the fractional occupancy of the distinguishable modifications in (b) to the fractional occupancy of the distinguishable modifications in the absence of said modulator;
wherein alteration of the amount of a distinguishable modification in the presence of said modulator as compared to in the absence of said modulator indicates that said modulator acts on the signaling pathway that causes said distinguishable modification.

11. The method of claim 10, wherein said modification-mediated signaling pathways are kinase pathways.

12. The method of claim 11, wherein said kinase pathways comprise proteins selected from the group consisting of: RSK, ERK, cMet, Glycogen Synthase, STAT1-5, Histone H3, INCEP, and EGFR.

13. The methods of claim 10, wherein said modulator is an inhibitor.

14. The method of claim 10, wherein said biological sample is selected from the group consisting of: cultured cells, harvested tissues, and clinical samples.

15. A method of identifying the dominant driver between at least two modification-mediated signaling pathways, wherein each of said signaling pathways causes at least one distinguishable modification on a protein, said method comprising the steps of:
a) determining the fractional occupancy of said at least one distinguishable modification caused by each signaling pathway in a biological sample in which said signaling pathways are operative;
b) quantifying the amount of said protein;
c) calculating the drive product and the drive ratio for said protein for said at least one distinguishable modification caused by each signaling pathway; and
d) calculating the difference between the extent of said distinguishable modifications of (c) and the extent of each of said distinguishable modifications in the absence of said treatment;
wherein the signaling pathway which results in a modification with the highest drive product and the lowest drive ratio is the dominant driver; or if two signaling pathways have similar drive products, then the signaling pathway with the lower drive ratio therebetween is the dominant driver.

16. The method of claim 15, wherein said distinguishable modifications occur at two separately modifiable residues of said protein.

17. The method of claim 15, wherein said modification-mediated signaling pathway is a kinase pathway.

18. The method of claim 16, wherein said distinguishable modifications are both phosphorylation.

19. The method of claim 15, wherein said biological sample is selected from the group consisting of: cultured cells, harvested tissues, and clinical samples.

20. A method of detennining which of at least two modification-mediated signaling pathways is more sensitive to a modulator comprising the steps of:
a) determining the fractional occupancy of modification of said at least one protein in each of said at least two modification-mediated signaling pathways in a biological sample;
b) quantifying the amount of said at least one protein per cell;
c) calculating the drive product and drive ratio for said at least one protein; and
d) calculating the difference between the drive product and drive ratio of (c) and the drive product and drive ratio of said protein in the absence of said modulator;
wherein the protein with the highest difference in the drive product and the lowest difference in the drive ratio is more sensitive therebetween to said modulator; or if two signaling pathways have similar differences in the drive products, then the signaling pathway with the lowest difference in the drive ratio is the more sensitive therebetween to said modulator.

21. The method of claim 20, wherein said modification-mediated signaling pathway is a kinase pathway.

22. The method of claim 21, wherein said distinguishable modifications are both phosphorylation.

23. The method of claim 20, wherein said biological sample is selected from the group consisting of: cultured cells, harvested tissues, and clinical samples.

24. A method of monitoring the progress in a patient of therapy using a modulator of a protein comprising at least one modifiable residue, said method comprising the steps of:
a) exposing said patient to said compound;
b) obtaining a biological sample comprising said protein from said patient;
c) determining the extent of modification of said at least one modifiable residue is said cells; and
d) comparing the extent of modification of said at least one modifiable residue to the extent of modification of said at least one modifiable residue in the absence of said compound.

25. The method of claim 24, wherein said modification-mediated signaling pathway is a kinase pathway.

26. The method of claim 25, wherein said kinase pathway comprises a protein selected from the group consisting of: RSK, ERK, cMet, Glycogen Synthase, STAT1-5, Histone H3, INCEP, and EGFR.

27. The method of claim 24, wherein said modulator is an inhibitor.

28. The method of claim 24, wherein said biological sample is selected from the group consisting of: a blood sample or a tumor biopsy.

29. A method of determining which of at least two modulators of a modification-mediation signaling pathway is more useful as a treatment for a condition characterized by aberrant signaling through said pathway comprising:
a) separately exposing at least two biological samples characterized by aberrant signaling through said pathway to each of said at least two modulators, wherein said biological samples comprise a protein that is modified by said modification-mediated signaling pathway;
b) separately determining the extent of modification of said protein;
c) separately quantifying the amount of said protein per cell;
d) calculating the drive products and drive ratios for said protein in the presence of each of said at least two modulators; and
e) calculating the differences between the drive products and drive ratios of (d) to the drive product and drive ratio for said protein in said cell in the absence of said modulator;
wherein the modulator that is associated with the highest difference in drive product and the lowest difference in drive ratio in (e) is more useful therebetween as a treatment for said condition.

30. The method of claim 29, wherein said modification-mediated signaling pathway is a kinase pathway.

31. The method of claim 30, wherein said kinase pathway comprises a protein selected from the group consisting of: RSK, ERK, cMet, Glycogen Synthase, STAT1-5, Histone H3, INCEP, and EGFR.

32. The method of claim 30, wherein said modulators are inhibitors.

33. The method of claim 30, wherein said biological sample is selected from the group consisting of: cultured cells, harvested tissues, and clinical samples.

34. A composition comprising at least one oligopeptide selected from SEQ ID NO: 1-54, wherein each of said polypeptides comprises at least one modification that renders it distinguishable by MS from the corresponding non-modified oligopeptide.

35. The composition of claim 34, wherein said modification is at least one amino-acid residue comprising ¹³C and/or ¹⁵N.

36. The composition of claim 34, wherein said modified amino-acid residues comprise an affinity tag.
